(19) United States
(12) Patent Application Publication
(10) Pub. No.: US 2010/0233198 A1
(43) Pub. Date: Sep. 16, 2010

(54) USE OF OLIGOSACCHARIDES CONTAINING N-ACETYLLACTOSAMINE FOR MATURATION OF IMMUNE RESPONSES IN NEONATES

(75) Inventors: Marie-Claire Fichot, Blonay (CH); Norbert Sprenger, Savigny (CH)

Correspondence Address: K&L Gates LLP, P.O. Box 1135, CHICAGO, IL, 60690, US

(73) Assignee: NESTEC S.A., Vevey (CH)

(21) Appl. No.: 12/741,997

(22) PCT Filed: Nov. 7, 2008
(86) PCT No.: PCT/EP08/65143

§ 371 Date: May 7, 2010

(30) Foreign Application Priority Data
Nov. 8, 2007 (EP) 07120265.9

(51) Int. Cl. A61K 39/00; A61K 31/702; A61K 35/74

(52) U.S. Cl. 424/184.1; 514/54; 424/93.4

(57) ABSTRACT

This invention relates to the use of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose in the manufacture of a medicament for infants or a therapeutic nutritional composition for infants for modulating immune responses in a neonatal infant. The invention extends to the use of such an oligosaccharide for modulating the immune system of a neonatal infant to promote the development in the first few weeks of the life of the infant of a beneficial intestinal microbiota comparable with that found in breast fed infants and for reducing the risk of subsequent development of allergy in the infant.

### FIELD OF THE INVENTION

This invention relates to the maturation of immune responses of neonatal infants by education of the immune system to avoid inappropriate immune responses.

### BACKGROUND TO THE INVENTION

Immediately before birth, the gastro-intestinal tract of a baby is thought to be sterile. During the normal process of birth, it encounters bacteria from the digestive tract, skin and environment of the mother and starts to become colonised. The faecal microbiota of a healthy breast-fed infant of age 2 to 4 weeks which may be taken as the optimum microbiota for this age group is dominated by Bifidobacteria species with some Lactobacillus species and lesser amounts of Bacteroides such as Bacteroides fragilis species, to the exclusion of potential pathogens such as Clostridia. After the completion of weaning at about 2 years of age, a pattern of gut microbiota that resembles the adult pattern becomes established.

The early stages of colonisation is a critical phase of adaptation to the gut's new environment, which provides most of the identified innate immune receptor ligands, such as lipopolysaccharides, at high concentrations. Gut innate immune recognition must therefore be closely regulated and educated during this period in order to avoid inappropriate stimulation.

In mice, intestinal epithelial cells respond within several hours of birth to exogenous bacterial compounds, such as endotoxins, to acquire toll like receptor tolerance to facilitate subsequent microbial colonization and the development of a stable intestinal host-microbe homeostasis (Lotz et al., 2006). It is hypothesised that similar postnatal processes also occur in human neonates. Dysfunction of the down-regulation of proinflammatory reactions which is necessary to allow colonisation to take place may lead to the development of enteric inflammation and disturbances in the colonisation process. This in turn could lead to a long-lasting battle between host and microbes, with negative consequences for health in the short and likely also at the long term both locally in the gut and even systemically.

It has already been suggested that this delayed or inappropriate colonisation may have specific consequences in terms of the subsequent development of the infant. For example, Fantuzzi et al have proposed that systemic low-grade inflammation and a sub-optimal gut microbiota may be implicated in the later development of obesity (Fantuzzi G. “Adipose tissue, adipokines, and inflammation” J Allergy Clin Immunol. 2005; 115:911-919).

In short, more and more evidence is emerging which suggests that the establishment of an appropriate intestinal microbiota early in life may be a significant factor in subsequent healthy development. Moreover, an inappropriate intestinal microbiota is likely to be accompanied by local inflammation in the gut and low-level systemic inflammation which has its own adverse consequences on general health.

Mother's milk is recommended for all infants. In addition to macro-nutrients, human milk is known to contain many bioactive nutrients, that is, compounds which may be categorised in broad terms as proteins, carbohydrate or fats but which have a specific function over and above their calorific value. One large class of such bioactive nutrients is human milk oligosaccharides, a group of over 100 oligosaccharides which are found in varying quantities in human milk but not in the same quantity or variety in bovine milk for example. The number and function of these various oligosaccharides are still being elucidated although certain of them have already been associated with, for example, reducing the ability of pathogens to adhere to host epithelial cells.

However, in some cases breast feeding is inadequate or unsuccessful for medical reasons or the mother chooses not to breast feed. Infant formulae have been developed for these situations. The vast majority of infant formulae are based on bovine milk which, as noted above, is not as rich in functional carbohydrates either in terms of diversity or content as human milk.

### SUMMARY OF THE INVENTION

The present inventors have surprisingly found that administration of oligosaccharides with lactose bound mono-, di- and/or poly-valent N-acetyl-lactosamine structures to the neonatal infant is particularly effective in the education of the immune system, promoting down-regulation of inappropriate pro-inflammatory responses during the establishment of the intestinal microbiota in the neonatal period with the result that inappropriate immune responses can likewise be down-regulated.

Accordingly, in a first aspect, the present invention provides the use of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose lacto-N-octaose, lacto-N-neooctaose, iso-lacto-N-octaose, para-lacto-N-octaose and lacto-N-decaose in the manufacture of a medicament for infants or a therapeutic nutritional composition for infants for maturation of immune responses in a neonatal infant.

In a second aspect, the invention provides the use of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose in the manufacture of a medicament for infants or a therapeutic nutritional composition for infants for modulating the immune system of a neonatal infant to promote the development in the first few weeks of the life of the infant of a beneficial intestinal microbiota comparable with that found in breast fed infants.

In a third aspect, the invention provides the use of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose in the manufacture of a medicament or therapeutic nutritional composition for administration to a neonatal infant for reducing the risk of subsequent development of allergy in the infant.

The invention extends to a method of promoting the maturation of immune responses in a neonatal infant by administering to a neonatal infant in need thereof a therapeutic amount of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

The invention further extends to a method of promoting the development in the first few weeks of the life of an infant of a beneficial intestinal microbiota comparable with that found in breast fed infants by administering to a neonatal infant in need thereof a therapeutic amount of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

Finally, the invention extends to a method of reducing the risk of subsequent development of allergy in an infant by administering to a neonatal infant in need thereof a therapeutic amount of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

Without wishing to be bound by theory, the present inventors believe that oligosaccharides with lactose bound mono-, di- and/or poly-valent N-acetyl-lactosamine structures in the intestinal lumen may act as a “decoy” for pro-inflammatory factors preventing them from binding to their natural ligands and thereby alleviating pro-inflammatory responses. Specifically, galectin-3 is a soluble β-galactoside-binding protein involved in various pro-inflammatory reactions, such as activation of neutrophils (e.g. IL8). Neutrophils participate in the innate immune response as a major phagocytic leukocyte recruited to infection sites. Galectin-3 can be released from gut epithelia to activate the immune system.

Studies with galectin-3 null mice (gal−/−) showed the regulatory role of galectin-3 to tune both innate and adaptive immune responses to pathogens (Bernardes et al., 2006). With a similar pathogen burden, gal−/− mice showed much lower inflammatory response, lower leukocyte infiltration into gut lamina propria and no gut necrosis as compared to the wildtype controls. Thus it can be concluded that absence of galectin-3 leads to milder inflammatory response.

Glycans terminating with galactose β-1,4 N-acetylglucosamine such as lacto-N-neotetraose, bind galectin-3. As previously shown for galectin-9, free galectin-binding glycans can interfere with galectin binding to target ligands thereby altering target ligand behaviour (Ohtsubo et al., 2005). The more N-acetyllactosamine units are polymerized the higher the affinity for galectin-3 and the stronger the decoy function

Further, gal3−/− mice showed a higher Th1 response manifested by higher IL12 and IFN-γ production by dendritic cells (Bernardes et al., 2006). T helper cells play a central role in adaptive immunity. Th1 cells are vital for cell-mediated immune responses, and Th2 cells promote humoral immunity. Th1 and Th2 responses are counter-regulative, that is, cytokines produced by Th1 cells inhibit Th2 function and vice versa. Th2-skewed immune response has been shown to be crucial for the maintenance of successful pregnancy and it also prevails at birth and during the first months of life. Postnatal exposure to microbial antigens elicits preferentially Th1 responses, which have been suggested to counterbalance Th2-polarized cytokine production in neonates. In the case of insufficient early Th1 responses, the production of Th2-type cytokines (IL-4, IL-5 and IL-13) is further propagated leading to IgE production and consequently to allergic disease. It follows that the decoy activity described above also helps to bias the immune system towards a Th1 response thus educating the immune system and reducing the risk of development of allergy early in life.

### DETAILED DESCRIPTION OF THE INVENTION

In this specification, the following terms have the following meanings:—

“beneficial intestinal microbiota comparable with that found in breast fed babies” means an intestinal microbiota dominated by appreciable populations of Bifidobacterium and Lactobacillus species to the exclusion of appreciable populations of such species as Bacteroides, Clostridia and Streptococci;

“neonatal infant” means an infant in the first two months of life.

All references to percentages are percentages by weight unless otherwise stated.

The invention relates to the use of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose. Preferred oligosaccharides are lacto-N-tetraose (LNT) and lacto-N-neotetraose (LNnT). LNT and LNnT may be synthesised chemically by enzymatic transfer of saccharide units from donor moieties to acceptor moieties using glycosyltransferases as described for example in U.S. Pat. No. 5,288,637. Alternatively, LNT and LNnT may be prepared by chemical conversion of keto-hexoses (e.g. fructose) either free or bound to an oligosaccharide (e.g. lactulose) into N-acetylhexosamine or an N-acetylhexosamine containing oligosaccharide as described in Wrodnigg, T. M.; Stutz, A. E. (1999) Angew. Chem. Int. Ed. 38:827-828. N-acetyllactosamine produced in this way may then be transferred to lactose as acceptor moiety.

The therapeutic nutritional composition is preferably an infant formula which contains an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose in an amount between 0.1 and 3 g/100 g composition on a dry weight basis.

An infant formula for use according to the present invention may contain a protein source in an amount of not more than 2.0 g/100 kcal, preferably 1.8 to 2.0 g/100 kcal. The type of protein is not believed to be critical to the present invention provided that the minimum requirements for essential amino acid content are met and satisfactory growth is ensured although it is preferred that over 50% by weight of the protein source is whey. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in whatever proportions are desired.

The proteins may be intact or hydrolysed or a mixture of intact and hydrolysed proteins. It may be desirable to supply partially hydrolysed proteins (degree of hydrolysis between 2 and 20%), for example for infants believed to be at risk of developing cows' milk allergy. If hydrolysed proteins are required, the hydrolysis process may be carried out as desired and as is known in the art. For example, a whey protein hydrolysate may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

The infant formula may contain a carbohydrate source. Any carbohydrate source conventionally found in infant formulae such as lactose, saccharose, maltodextrin, starch and mixtures thereof may be used although the preferred source of carbohydrates is lactose. Preferably the carbohydrate sources contribute between 35 and 65% of the total energy of the formula.

The infant formula may contain a source of lipids. The lipid source may be any lipid or fat which is suitable for use in infant formulas. Preferred fat sources include palm olein, high oleic sunflower oil and high oleic safflower oil. The essential fatty acids linoleic and α-linolenic acid may also be added as may small amounts of oils containing high quantities of preformed arachidonic acid and docosahexaenoic acid such as fish oils or microbial oils. In total, the fat content is preferably such as to contribute between 30 to 55% of the total energy of the formula. The fat source preferably has a ratio of n-6 to n-3 fatty acids of about 5:1 to about 15:1; for example about 8:1 to about 10:1.

The infant formula may also contain all vitamins and minerals understood to be essential in the daily diet and in nutritionally significant amounts. Minimum requirements have been established for certain vitamins and minerals. Examples of minerals, vitamins and other nutrients optionally present in the infant formula include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. Minerals are usually added in salt form. The presence and amounts of specific minerals and other vitamins will vary depending on the intended infant population.

If necessary, the infant formula may contain emulsifiers and stabilisers such as soy lecithin, citric acid esters of mono- and di-glycerides, and the like.

The infant formula preferably further contains at least one prebiotic in an amount of 0.3 to 10%. A prebiotic is a non-digestible food ingredient that beneficially affects the host by selectively stimulating the growth and/or activity of one or a limited number of bacteria in the colon, and thus improves host health. Such ingredients are non-digestible in the sense that they are not broken down and absorbed in the stomach or small intestine and thus pass intact to the colon where they are selectively fermented by the beneficial bacteria. Examples of prebiotics include certain oligosaccharides, such as fructooligosaccharides (FOS) and galactooligosaccharides (GOS). A combination of prebiotics may be used such as 90% GOS with 10% short chain fructo-oligosaccharides such as the product sold under the trade mark Raftilose® or 10% inulin such as the product sold under the trade mark Raftiline®. A particularly preferred combination of prebiotics is 70% short chain fructo-oligosaccharides and 30% inulin.

The infant formula may also comprise at least one probiotic bacterial strain. A probiotic is a microbial cell preparation or components of microbial cells with a beneficial effect on the health or well-being of the host. Suitable probiotic bacterial strains include Lactobacillus rhamnosus ATCC 53103 obtainable from Valio Oy of Finland under the trade mark LGG, Lactobacillus rhamnosus CGMCC 1.3724, Lactobacillus paracasei CNCM I-2116, the strain of Lactobacillus reuteri sold by BioGaia A.B under the trade mark Reuteri, Streptococcus salivarius DSM 13084 sold by BLIS Technologies Limited of New Zealand under the designation K12, Bifidobacterium lactis CNCM I-3446 sold inter alia by the Christian Hansen company of Denmark under the trade mark Bb12, Bifidobacterium longum ATCC BAA-999 sold by Morinaga Milk Industry Co. Ltd. of Japan under the trade mark BB536, the strain of Bifidobacterium breve sold by Danisco under the trade mark Bb-03, the strain of Bifidobacterium breve sold by Morinaga under the trade mark M-16V, the strain of Bifidobacterium infantis sold by Procter & Gamble Co. under the trade mark Bifantis and the strain of Bifidobacterium breve sold by Institut Rosell (Lallemand) under the trade mark R0070 in an amount between 10e3 and 10e12 cfu/g powder, more preferably between 10e7 and 10e12 cfu/g powder.

The infant formula may optionally contain other substances which may have a beneficial effect such as lactoferrin, nucleotides, nucleosides, and the like.

The infant formula may be prepared in any suitable manner. For example, they may be prepared by blending together the protein, the carbohydrate source, and the fat source in appropriate proportions. If used, the emulsifiers may be included at this point. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently about 50° C. to about 80° C. to aid dispersal of the ingredients. Commercially available liquefiers may be used to form the liquid mixture. The N-acetyl-lactosamine and/or an oligosaccharide containing N-acetyl-lactosamine will be added at this stage if the final product will be in liquid form. If the final product is to be a powder, the oligosaccharides may likewise be added at this stage if desired. The liquid mixture is then homogenised; for example in two stages.

The liquid mixture may then be thermally treated to reduce bacterial loads, by rapidly heating the liquid mixture to a temperature in the range of about 80° C. to about 150° C. for about 5 seconds to about 5 minutes, for example. This may be carried out by steam injection, autoclave or by heat exchanger; for example a plate heat exchanger.

Then, the liquid mixture may be cooled to about 60° C. to about 85° C.; for example by flash cooling. The liquid mixture may then be again homogenised; for example in two stages at about 10 MPa to about 30 MPa in the first stage and about 2 MPa to about 10 MPa in the second stage. The homogenised mixture may then be further cooled to add any heat sensitive components; such as vitamins and minerals. The pH and solids content of the homogenised mixture are conveniently adjusted at this point.

The homogenised mixture is transferred to a suitable drying apparatus such as a spray drier or freeze drier and converted to powder. The powder should have a moisture content of less than about 5% by weight. The oligosaccharide may be added at this stage by dry-mixing along with the probiotic bacterial strain(s) if used

If a liquid product is preferred, the homogenised mixture may be sterilised then aseptically filled into suitable containers or may be first filled into the containers and then retorted.

In another embodiment, the composition may be a supplement including an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose in an amount sufficient to achieve the desired effect. Preferably the daily dose of the oligosaccharide is between 0.1 and 3 g. The amount of oligosaccharide to be included in the supplement will be selected accordingly depending upon how the supplement is to be administered. For example, if the supplement is to be administered twice a day, each supplement may contain between 0.05 and 1.5 g. The supplement should be in a form suitable for administration to neonatal infants and will contain only those adjuvants and excipients suitable for this age group.

The invention will now be further illustrated by reference to the following example:—

### Example 1

An example of the composition of a suitable infant formula to be used in the present invention is given below

| Nutrient | per 100 kcal | per litre |
|---|---|---|
| Energy (kcal) | 100 | 670 |
| Protein (g) | 1.83 | 12.3 |
| Fat (g) | 5.3 | 35.7 |
| Linoleic acid (g) | 0.79 | 5.3 |
| α-Linolenic acid (mg) | 101 | 675 |
| Lactose (g) | 11.2 | 74.7 |
| Minerals (g) | 0.37 | 2.5 |
| Na (mg) | 23 | 150 |
| K (mg) | 89 | 590 |
| Cl (mg) | 64 | 430 |
| Ca (mg) | 62 | 410 |
| P (mg) | 31 | 210 |
| Mg (mg) | 7 | 50 |
| Mn (μg) | 8 | 50 |
| Se (μg) | 2 | 13 |
| Vitamin A (μg RE) | 105 | 700 |
| Vitamin D (μg) | 1.5 | 10 |
| Vitamin E (mg TE) | 0.8 | 5.4 |
| Vitamin K1 (μg) | 8 | 54 |
| Vitamin C (mg) | 10 | 67 |
| Vitamin B1 (mg) | 0.07 | 0.47 |
| Vitamin B2 (mg) | 0.15 | 1.0 |
| Niacin (mg) | 1 | 6.7 |
| Vitamin B6 (mg) | 0.075 | 0.50 |
| Folic acid (μg) | 9 | 60 |
| Pantothenic acid (mg) | 0.45 | 3 |
| Vitamin B12 (μg) | 0.3 | 2 |
| Biotin (μg) | 2.2 | 15 |
| Choline (mg) | 10 | 67 |
| Fe (mg) | 1.2 | 8 |
| I (μg) | 15 | 100 |
| Cu (mg) | 0.06 | 0.4 |
| Zn (mg) | 0.75 | 5 |
| LNnT (mg) | 37 | 250 |

1. A method for assisting in the maturation of immune responses in a neonatal infant comprising the step of administering a composition to the neonatal infant comprising an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

2. A method for modulating the immune system of a neonatal infant to promote the development in the first few weeks of the life of the infant of a beneficial intestinal microbiota comparable with that found in breast fed infants comprising the step of administering a composition comprising an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

3. A method for reducing the risk of subsequent development of allergy in an infant comprising the step of administering to the infant a composition comprising an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

4. The method of claim 1, wherein the oligosaccharide is lacto-N-tetraose or lacto-N-neotetraose.

5. The method of claim 1, wherein the composition is an infant formula.

6. The use of claim 5, wherein the infant formula contains from 0.1 to 3 g of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose per 100 g formula.

7. The method of claim 5, where the infant formula comprises a probiotic bacterial strain in an amount of from 10e3 to 10e12 cfu/g formula on a dry weight basis.

8. The method of claim 5, wherein the infant formula comprises at least one prebiotic in an amount of from 0.3 to 10% by weight of the formula.

9. The method of claim 1, wherein the composition is a supplement which comprises from 0.1 to 3 g of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose per daily dose.

10. The method of claim 1, wherein the composition is administered to the infant immediately after delivery and thereafter for at least 2 months.

11. The method of claim 1, wherein the composition is selected from the group consisting of medicament and therapeutic nutritional composition.

12. The method of claim 2, wherein the oligosaccharide is lacto-N-tetraose or lacto-N-neotetraose.

13. The method of claim 2, wherein the composition is an infant formula.

14. The use of claim 13, wherein the infant formula contains from 0.1 to 3 g of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose per 100 g formula.

15. The method of claim 13, where the infant formula comprises a probiotic bacterial strain in an amount of from 10e3 to 10e12 cfu/g formula on a dry weight basis.

16. The method of claim 13, wherein the infant formula comprises at least one prebiotic in an amount of from 0.3 to 10% by weight of the formula.

17. The method of claim 2, wherein the composition is a supplement which comprises from 0.1 to 3 g of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose per daily dose.

18. The method of claim 2, wherein the composition is administered to the infant immediately after delivery and thereafter for at least 2 months.

19. The method of claim 2, wherein the composition is selected from the group consisting of medicament and therapeutic nutritional composition.

20. The method of claim 3, wherein the oligosaccharide is lacto-N-tetraose or lacto-N-neotetraose.

21. The method of claim 3, wherein the composition is an infant formula.

22. The use of claim 21, wherein the infant formula contains from 0.1 to 3 g of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose per 100 g formula.

23. The method of claim 21, where the infant formula comprises a probiotic bacterial strain in an amount of from 10e3 to 10e12 cfu/g formula on a dry weight basis.

24. The method of claim 21, wherein the infant formula comprises at least one prebiotic in an amount of from 0.3 to 10% by weight of the formula.

25. The method of claim 3, wherein the composition is a supplement which comprises from 0.1 to 3 g of an oligosaccharide selected from the group consisting of lacto-N-tetraose, lacto-N-neotetraose, lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose per daily dose.

26. The method of claim 3, wherein the composition is administered to the infant immediately after delivery and thereafter for at least 2 months.

27. The method of claim 3, wherein the composition is selected from the group consisting of medicament and therapeutic nutritional composition.
